SEQUENCE LISTING (2 sequences)

SEQ ID NO: 1 - DNA, 20 bp, Artificial Sequence
  Synthetic Construct - synthetic polynucleotide (ligand)
ttgaggtgcatgtttgtgcc

SEQ ID NO: 2 - DNA, 20 bp, Artificial Sequence
  Synthetic Construct - synthetic polynucleotide (anti ligand)
ggcacaaacatgcacctcaa

(19) United States
(12) Patent Application Publication
(10) Pub. No.: US 2013/0309781 A1
(43) Pub. Date: Nov. 21, 2013

(54) NOVEL ADHESIVE SURFACES FOR THE IMMOBILIZATION OF LIGANDS

(71) Applicants: Benjamin Corgier, Saint Etienne Des Oullieres (FR); Gaelle Legoff, Cambridge, MA (US); Celine Mandon, Villeurbanne (FR); Loic Blum, Caluire et Cuire (FR); Christophe Marquette, Villeurbanne (FR)

(72) Inventors: Benjamin Corgier, Saint Etienne Des Oullieres (FR); Gaelle Legoff, Cambridge, MA (US); Celine Mandon, Villeurbanne (FR); Loic Blum, Caluire et Cuire (FR); Christophe Marquette, Villeurbanne (FR)

(73) Assignees: CENTRE NATIONAL DE LA RECHERCHE SCIENTIFIQUE, Paris (FR); INSTITUT NATIONAL DES SCIENCES APPLIQUEES DE LYON, Villeurbanne (FR); UNIVERSITE CLAUDE BERNARD, Villeurbanne (FR)

(21) Appl. No.: 13/978,061

(22) PCT Filed: Jan. 12, 2012
(86) PCT No.: PCT/FR12/50085

§ 371 Date: Jul. 30, 2013

(30) Foreign Application Priority Data
Jan. 14, 2011 (FR) 11/50308

(51) Int. Cl. G01N 33/543; G01N 33/531

(52) U.S. Cl. CPC G01N 33/543; G01N 33/531
USPC 436/501; 422/69; 156/69; 427/299

(57) ABSTRACT

A complex including: a support provided with at least two faces one of which is provided with a coating of an adhesive, at least one ligand, said ligand being immobilized on the adhesive surface. The ligand implemented within the framework of the present invention is chosen from among proteins, peptides, antibodies, nucleic acids, sugars or oligosaccharides, toxins, pesticides, hormones, herbicides, fungicides, neurotransmitters.

The present invention relates to a novel complex comprising at least one ligand immobilized on an adhesive coated on one of the faces of the support.

The use of ligands immobilized on a support is common, especially in the field of diagnostic tests or in the biotechnological field. These systems allow the use of a large number of molecules, as in the case of the chips used for DNA or RNA analysis.

Immobilization of ligands on a support has been widely described (Sambrook et al. 1989). The immobilization methods may prove to be difficult, expensive, long and partially effective. They are based on adsorption, ionic or covalent bonding, or the trapping of ligands in a matrix of gel or polymer type.

Methods have been developed, and thus patent application WO 2005/114 417 describes the use of a layer of globular proteins applied to an adhesive support, a crosslinking agent then being applied to the layer of globular proteins, the protein catalysts being immobilized at the surface by reaction with the crosslinking agent.

Document WO 03/072 752 describes protein chips produced on a rigid substrate bearing a hydrophobic and polymeric layer of polyvinylidene difluoride (PVDF) enabling the immobilization of proteins in dry form, a high spot density and having a high signal-to-noise ratio.

The present invention makes it possible to overcome the drawbacks described above by means of the use of a complex comprising a support having at least two faces, one of which is provided with a coating of an adhesive and of at least one ligand immobilized on said adhesive surface.

In the context of the present invention, the ligand deposited on the adhesive surface may be of protein nature: protein, oligopeptide, polypeptide, antibody, of nucleic acid nature: DNA, RNA, oligonucleotide (i.e. primer), it may also be sugars (oligosaccharides or polysaccharides) or small molecules, which are optionally synthetic, such as toxins, pesticides, hormones, herbicides, fungicides or neurotransmitters.

Thus, when the ligand is of protein type, no pretreatment of the adhesive surface is necessary.

The support used may consist of a glass or plastic plate, a polyester film or any other material that may be coated with an adhesive.

In one particular embodiment of the invention, the support is provided with a coating of an adhesive on both of its faces (FIG. 1).

The adhesive is a common adhesive polymer, which may have an activated surface. Examples that will be mentioned include unreactive adhesives such as styrene-butadiene copolymers, nitrile rubbers, poly(vinyl acetate) and polymers thereof, polyvinyl acetals, pressure-sensitive adhesives such as polyacrylates, silicone rubbers, poly(vinyl ethers), or alternatively reactive adhesives such as two-pack polyurethane adhesives, epoxy adhesives, anaerobic acrylics or cyanoacrylates.

For example, the following materials have been used:
3M 7966WDL (adhesive 3M™ 200 MP)
5 Stars Double Sided Display Tape Polypropylene, 3M™ Optically Clear Overlaminating Film 76991, Ultra Clear Removable Overlaminating Film 76991.

The ligands are chosen so as to interact specifically with defined antiligands. Depending on the nature of the substrate and the immobilization strategy (via affinity, covalent, etc.), the ligands may be functionalized so as to obtain better immobilization.

Various ligands may be matrixed simultaneously onto the same support and the design of the matrix may furthermore include several replicas of the same probe (FIG. 1).

For protein ligands, no pretreatment of the surface of the adhesive is necessary, and the ligands may be deposited (spotting step) directly onto the surface.

For oligonucleotide ligands, a step of surface pretreatment using a multifunctional crosslinking agent (preferably glutaraldehyde) may be performed in order to improve the activity of the immobilized ligands.

Thus, according to another aspect, a subject of the invention is a complex comprising a support, one of the faces of which is provided with a coating of an adhesive, said adhesive surface being functionalized, for example via the action of a crosslinking agent. In one particular embodiment, the ligand is also functionalized.

A subject of the present invention is also a process for preparing the complexes described above.

In one particular embodiment of the invention, the ligands are diluted in a buffer, for example a saline buffer (selected according to the nature of the biomolecules used as ligands), and deposited on the surface of the coated support using, for example, a piezoelectric spotter of noncontact type or by immersing the coated support in a solution comprising the ligands.

When the ligands in solution are deposited in the form of drops, the size of the deposited drops and consequently the size of the spots formed on the surface (50-1000 μm), the spot density (1-25 per mm²) and the format of the matrix may be modified as a function of the desired application field and of the nature of the biomolecules.

A step for functionalizing the support may be performed before depositing the ligands, for instance treatment of the adhesive surface with a crosslinking agent such as glutaraldehyde.

Depending on the application field under consideration, a post-treatment step may be performed after the steps of depositing the ligand and optionally of drying at room temperature. This post-treatment step may consist in heating (for example at 163° C. for one minute), washing (saline buffers, etc.) and/or saturating (in order to reduce the background noise) the complex.

The complexes according to the invention may be used for the preparation of devices especially for analysis.

Such devices comprise a substantially rigid support comprising at least one well delimiting an internal cavity comprising at least two apertures, at least one of them being sealed by the complex according to the invention.

Such devices may take the form of 12-, 24- or 96-well plates or alternatively of microfluidic networks commonly used in the field of diagnostic tests or in the biotechnological field.

The process for preparing such devices consists in sealing the complex according to the invention to the support so as to close at least one of said apertures of the cavity, the adhesive surface comprising the ligands being oriented toward the interior of the Cavity.

The adhesive surface modified with the ligands may be readily assembled with materials of varied types for the purpose of generating ready-to-use analytical tools (FIGS. 2 and 3).

For example, the adhesive support may be assembled with a bottomless 96-well plate in order to generate a solid-bottomed plate conventionally used for analytical purposes.

The adhesive support may also be interfaced with various microfluidic networks, composed of channels, flow cells or mixers. The assembled microfluidic part may consist of any material available for this type of application (glass, silicon, plastics, and other polymeric materials).

A subject of the present invention is also a process for detecting and/or quantifying an antiligand, comprising the use of a device or of a complex as described above. Said complex or device is placed in contact with a sample liable to contain an antiligand under conditions enabling interaction between the ligand and the antiligand, optionally a labeled detection molecule is added and, finally, the signals generated by the interaction between the ligand and the antiligand are detected and/or quantified.

In such a process, the antiligand may originate from a biological sample such as serum, blood or plasma, an environmental sample such as a sample of water, gas, air or soil, or a sample originating from the agrifood industry such as food.

In addition to the critical step consisting of the ligand/antiligand interaction, one or more steps may be added according to the imperatives inherent in the strategies for labeling and detecting the interaction (FIG. 4).

In this case, e.g. if an additional labeling step is required (e.g. interaction between the biotin group of a target and a streptavidin molecule), two strategies may be used:

a Two-Step Protocol:
1) incubation of the antiligand solution (ligand/antiligand interaction);
2) incubation of a solution of labeled molecules (e.g. conjugated streptavidin);

a One-Step Protocol:
1) incubation of a single premixed solution containing both the antiligands and the labeled molecules.

As a function of the application and of the requirements, various detection methods may be used, for example:
colorimetry using a label, for instance:
   indicator system with alkaline phosphatase/BCIP;
   indicator system with horseradish peroxidase-ABTS;
   gold particles, etc.;
chemiluminescence using a label as follows:
   horseradish peroxidase—system with chemiluminescent substrate;
   alkaline phosphatase—system with chemiluminescent substrate.

Radiography or detection and/or quantification of radioactivity may also be used.

### DESCRIPTION OF THE FIGURES

FIG. 1: schematic representation of the complex according to the invention, showing (A) the support (S), one surface of which is coated with adhesive (ad) on which ligands (L) are immobilized; (B) the support (S), two faces of which are coated with adhesive (ad), the ligands (L) being immobilized on one of the two faces.

FIG. 2: schematic representation of the assembly of devices according to the invention (A): a microfluidic network, (B) a 96-well plate.

FIG. 3: schematic representation of the assembly of a microfluidic network according to the invention.

FIG. 4: schematic representation of the process for detecting an antiligand in a sample.

FIG. 5: curve presenting the correlation between the intensity of the detected signal and the concentration of oligonucleotide (antiligand) in the sample.

FIG. 6: curve presenting the correlation between the intensity of the detected signal and of the concentration of CRP (antiligand) in the sample.

FIG. 7: curve presenting the correlation between the intensity of the detected signal and the concentration of CRP (antiligand) in the sample.

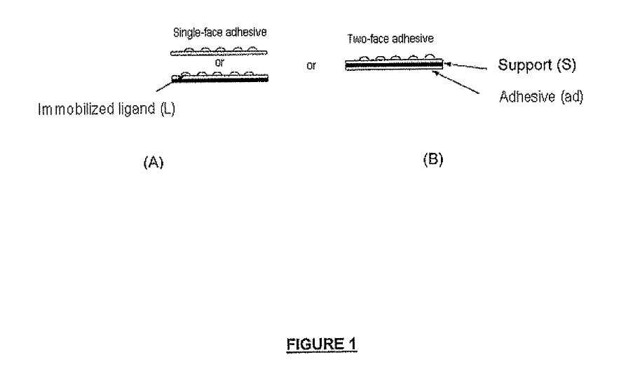

### EXAMPLES

### Example 1

### Matrix of Oligonucleotides on Adhesive Microtitration Plates (for the Quantitative Detection of Antiligand)

Using a pair of synthetic oligonucleotides of complementary sequences as ligand and antiligand, it was shown that the present invention can be applied to the analysis and to the quantitative detection of oligonucleotide sequences.

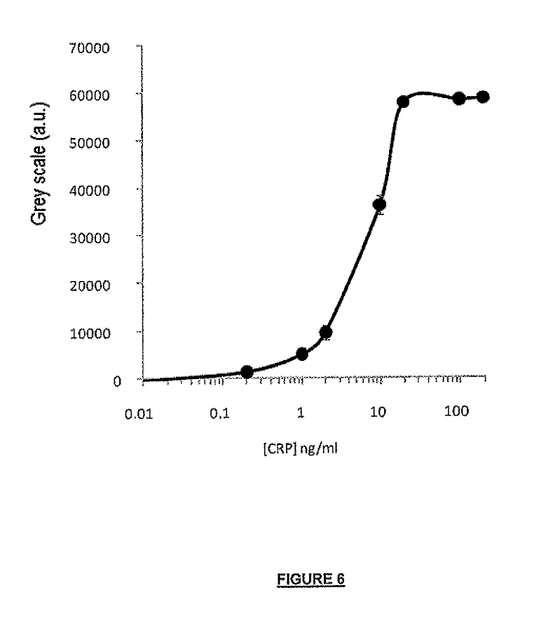

The ligands were spotted onto the surface of the adhesive support and hybridized with an oligonucleotide of complementary sequence (antiligand). A revealing system of biotin-streptavidin alkaline phosphatase type was used for the colorimetric revelation of the signal. A correlation between the intensity of the measured signal and the concentration of antiligand was demonstrated (FIG. 5).

Spotting of the Probes

### Pretreatment

A support 3M 7966WDL was pretreated with a 1% glutaraldehyde solution in 0.1M pH 5 phosphate buffer for 1 hour at 37° C. The supports were then washed with distilled water in order to be ready for use for immobilizing the probes.

### Spotting

The ligands (synthetic oligonucleotides (SEQ ID No. 1, 5′-amino modification) were diluted in saline acetate buffer (0.1M acetate, 0.1M KCl, 0.25 mg/mL bromophenol blue, pH=5.5) in order to reach a final concentration of 50 μmol·L⁻¹. This solution was spotted onto the surface of an adhesive 3M 7966WDL using a piezoelectric spotter (BioChip Arrayer BCA1, PerkinElmer). The substrate was dried in the open air and at room temperature. The spots produced have a diameter of about 100 μm and the spot density ranges from 1 to 25 spots per mm².

### Post-Treatment

After matrixing the ligands and drying the support at room temperature, the post-treatment was performed as follows: the supports are heated at 163° C. for 1 minute, and then washed by addition of PBS buffer and then incubated at 37° C. for 15 minutes with PBSTA buffer (0.1M phosphate, 0.5M NaCl, pH=7.4, 0.1% v/v Tween 20, 1% w/v BSA) in order to saturate the surface.

Assembly

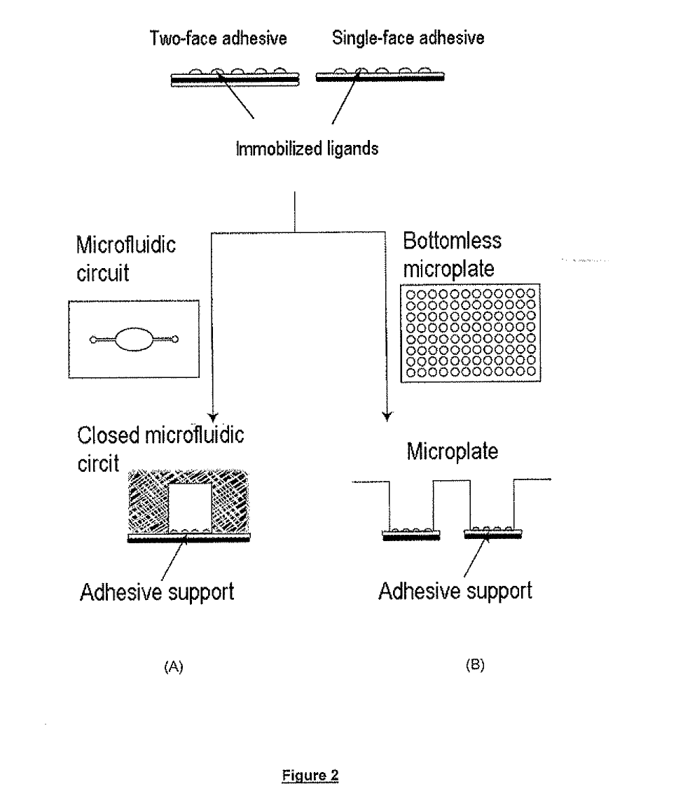

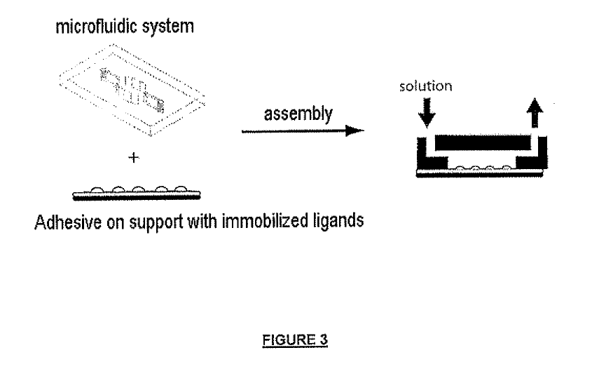

The spotted adhesives are then assembled with a bottomless 96-well plate in order to generate a solid-bottomed plate of standard use. The adhesive properties of the support are essential here so as to enable easy assembly performed by exerting a gentle pressure on the two parts placed one against the other (FIG. 2).

Test

Solutions of antiligands (SEQ ID No. 2) at different concentrations mixed with the alkaline phosphatase-streptavidin conjugate (1 μg·mL⁻¹) were prepared in a PBSTA buffer. 200 μl of the resulting solution were placed in each well and the assembly was then incubated on the spotted surface for 30 minutes at 37° C. The adhesive supports were then washed at room temperature with 1 mL of PBS solution.

Revelation 100 μL of a solution of BCIP/NBT (4-bromo-5-chloroindolyl phosphate/nitro-blue tetrazolium) were added to the wells of the plate and incubated at 37° C. in order to reveal the signal (about 30 minutes). The wells were then washed with 1 mL of PBS.

Once the bottoms of the wells were dry, acquisition of an image was formed using a horizontal office scanner (HP). A correlation between the intensity of the measured signal and the concentration of antiligand was demonstrated (FIG. 5).

### Example 2

### Protein Matrix on Adhesive Plate for Immunotest of Sandwich Type Applied to Point-of-Care Diagnosis

In the case where the adhesive supports are functionalized with microarrays of proteins (ligands), the present invention allows the performance of quantitative serological tests, which may be used for applications of point-of-care diagnostic type.

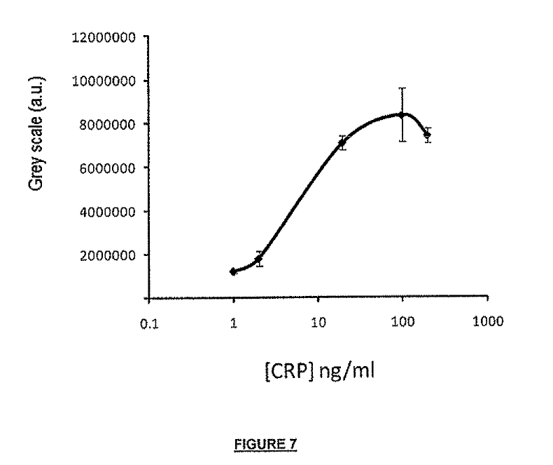

In order to evaluate the reliability of such a quantitative test, the following system was used: anti-CRP antibodies were immobilized on the support for the purpose of serving as probes for the detection of CRP using the corresponding targeted biotinylated antibodies. A revealing system of biotin-streptavidin alkaline phosphatase type was used for the calorimetric revelation of the signal. A correlation between the intensity of the measured signal and the concentration of target antibody (antiligand) was demonstrated (FIG. 6).

Spotting of the Ligands

Spotting

A solution of anti-CRP antibody at a concentration of 500 μg·mL⁻¹ in acetate buffer (0.1 M acetate, 0.1 M KCl, 0.25 mg/mL bromophenol blue, pH=5.5) was spotted onto the surface of an adhesive of 3M 7966WDL type using a piezoelectric spotter (BioChip Arrayer BCA1, PerkinElmer). No pretreatment of the surface is required. The surface was dried for 30 minutes at room temperature. The spots produced have a diameter of about 100 μm and the spot density may range from 1 to 25 per mm².

Post-Treatment

Following the matrixing of the microarray and drying at room temperature, the post-treatment is performed as follows: the supports are heated at +163° C. for 1 minute, then washed by addition of PBS buffer and then incubated at 37° C. for 15 minutes with PBSTA buffer (0.1M phosphate, 0.5M NaCl, pH=7.4, 0.1% v/v Tween 20, 1% w/v BSA) in order to saturate the surface.

Assembly

The spotted adhesives are then assembled with a bottomless 96-well plate in order to generate a solid-bottomed plate of standard use. The adhesive properties of the support are essential here so as to enable ready assembly performed by exerting a gentle pressure on the two parts placed one against the other (FIG. 2).

Test

Mixed solutions of target protein (CRP), of biotin-antibody conjugate (several concentrations) and of alkaline phosphatase-streptavidin conjugate (1 μg·mL⁻¹) were prepared in PBSTA buffer. 200 μL of the resulting solution were deposited in each well, and the whole was then incubated at 37° C. for 30 minutes. The adhesive supports were then washed at room temperature with 1 mL of PBS solution.

Revelation

100 μL of a solution of BCIP/NBT (4-bromo-5-chloroindolyl phosphate/nitro-blue tetrazolium) were added to the wells of the plate and incubated at 37° C. in order to reveal the signal (about 30 minutes). The wells were then washed with 1 mL of PBS.

Once the bottoms of the wells were dry, acquisition of an image was performed using a horizontal office scanner (HP). Examples of resulting images are presented in FIG. 6.

### Example 3

### Protein Matrix on Adhesive Support for Immunotest of Sandwich Type Applied to the Point-of-Care Diagnosis in an Assembled Microfluidic System

Spotting of the Ligands

Spotting

An anti-CRP antibody solution at a concentration of 500 μg·mL⁻¹ in an acetate buffer (0.1M acetate, 0.1M KCl, 0.25 mg/mL bromophenol blue, pH 5.5) was spotted onto the surface of an adhesive of 3M 7966WDL type using a piezoelectric spotter (BioChip Arrayer BCA1, PerkinElmer). No pretreatment of the surface is required. The surface was dried for 30 minutes at room temperature. The spots produced have a diameter of about 100 μm and the spot density may range from 1 to 25 per mm².

Post-Treatment

Following matrixing of the microarray and drying at room temperature, the post-treatment is performed as follows: the supports are heated at +163° C. for 1 minute and then washed by addition of PBS buffer and then incubated at 37° C. for 15 minutes with PBSTA buffer (0.1 M phosphate, 0.5 M NaCl, pH 7.4, 0.1% v/v Tween 20, 1% w/v BSA) in order to saturate the surface.

Assembly

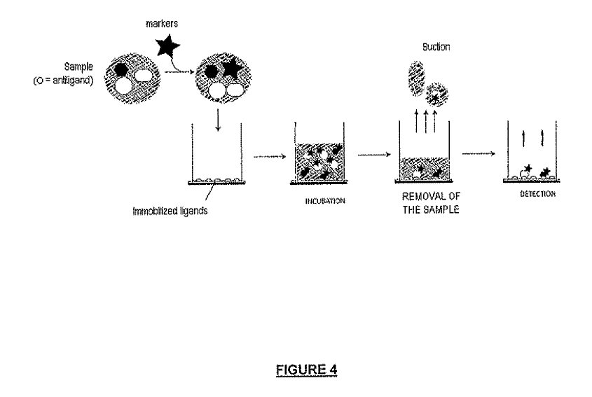

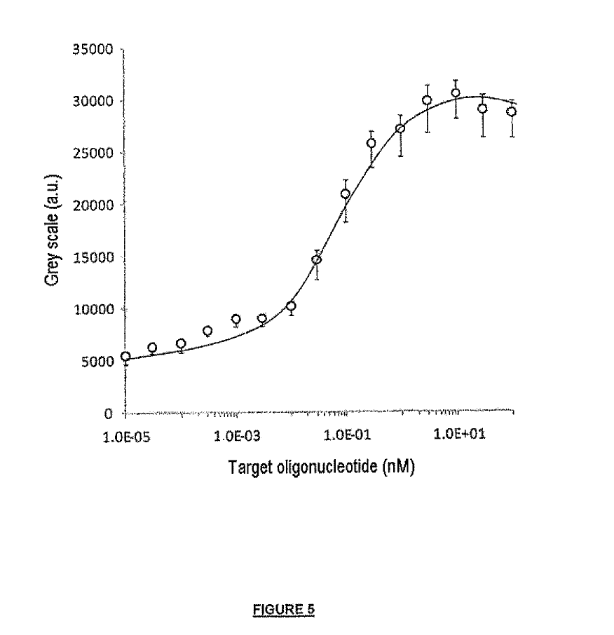

The adhesive support of matrixed proteins was assembled with a prefabricated microfluidic system consisting of PVC/3M 7966WDL microchannels or of glass microchannels obtained by etching. The adhesive properties of the support are essential here in order to enable ready assembly performed by exerting a gentle pressure on the two parts placed one against the other (FIG. 3).

Test

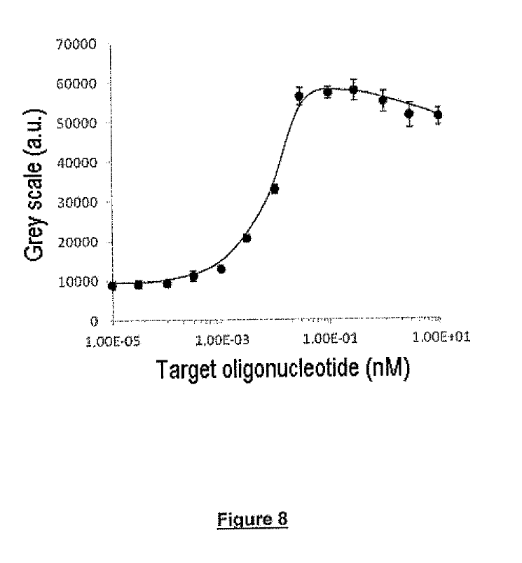

Mixed solutions of target protein (CRP, antiligand), of biotin-antibody conjugate (several concentrations) and of streptavidin-alkaline phosphatase conjugate (1 μg·mL⁻¹) were prepared in PBSTA buffer. 50 μL of the resulting solution were injected into the microfluidic network (FIG. 7) and the assembly was then incubated for one hour at 37° C. The assembled microfluidic system was then washed with 200 mL of PBS buffer.

Revelation

50 μL of a solution of BCIP/NBT (4-bromo-5-chloroindolyl phosphate/nitro-blue tetrazolium) were injected into the assembled microfluidic system and incubated at 37° C. in order to reveal the signal (about 30 minutes). The channels were then washed with 1 mL of PBS. Once the assembly was dried, acquisition of an image of the assembled microfluidic system was performed using a horizontal office scanner (HP). A correlation between the intensity of the measured signal and the concentration of antiligand was demonstrated (FIG. 7).

### Example 4

### Matrix of Oligonucleotides on Adhesive Microtitration Plates (for the Quantitative Detection of Antiligand)

The present invention may be applied to the analysis and the quantitative detection of oligonucleotide sequences (cf. Example 1).

In the present example, the ligands were spotted onto the surface of the adhesive support and hybridized with an oligonucleotide of complementary sequence (antiligand). A revealing system of biotin-streptavidin alkaline phosphatase type was used for the colorimetric revelation of the signal.

Spotting of the Probes

### Pretreatment

A 3M 7966WDL support was pretreated using a 1% solution of glutaraldehyde in 0.1 M pH 5 phosphate buffer for 1 hour at 37° C. The supports were then washed with distilled water in order to be ready for use for immobilization of the probes.

### Spotting

The ligands (synthetic oligonucleotides (SEQ ID No. 1, 5′-amino modification) were diluted in saline acetate buffer (0.1 M acetate, 0.1 M KCl, 0.25 mg/mL bromophenol blue, pH=5.5) in order to reach a final concentration of 50 μmol·L⁻¹. This solution was spotted onto the surface of a 3M 7966WDL adhesive using a piezoelectric spotter (BioChip Arrayer BCA1, PerkinElmer). The substrate was dried in the open air and at room temperature. The spots produced have a diameter of about 100 μm and the spot density ranges from 1 to 25 spots per mm².

Assembly

The spotted adhesives were then assembled with a bottomless 96-well plate in order to generate a solid-bottomed plate of standard use. The adhesive properties of the support are essential here in order to enable ready assembly performed by exerting a gentle pressure on the two parts placed one against the other (FIG. 2).

Test

Solutions of antiligands (SEQ ID No. 2) at different concentrations mixed with the alkaline phosphatase-streptavidin conjugate (1 μg·mL⁻¹) were prepared in a PBSTA buffer. 200 μL of the resulting solution were deposited in each well, and the whole was then incubated on the spotted surface for 30 minutes at 37° C. The adhesive supports were then washed at room temperature with 1 mL of PBS solution.

Revelation

100 μL of a solution of BCIP/NBT (4-bromo-5-chloro-indolyl phosphate/nitro-blue tetrazolium) were added to the wells of the plate and incubated at 37° C. in order to reveal the signal (about 30 minutes). The wells were then washed with 1 mL of PBS.

Once the bottoms of the wells were dry, acquisition of an image was performed using a horizontal office scanner (HP). A correlation between the intensity of the measured signal and the concentration of antiligand was demonstrated (FIG. 8).

1. A complex comprising:
a support provided with at least two faces, one of which is provided with a coating of an adhesive,
at least one ligand,
said ligand being immobilized on the adhesive surface, wherein the adhesive is chosen from pressure-sensitive adhesives such as polyacrylates, silicone gums, polyvinyl ethers, or unreactive adhesives such as styrene-butadiene copolymers, nitrile rubbers, poly(vinyl acetate) and polymers thereof, polyvinyl acetals, or reactive adhesives such as two-pack polyurethane adhesives, epoxy adhesives, anaerobic acrylics or cyanoacrylates.

2. The complex as claimed in claim 1, wherein the ligand is chosen from proteins, peptides, antibodies, nucleic acids, sugars or oligosaccharides, toxins, pesticides, hormones, herbicides, fungicides and neurotransmitters.

3. The complex as claimed in claim 1, said adhesive surface being functionalized.

4. The complex as claimed in claim 1, in which the ligand is functionalized.

5. The complex as claimed in claim 1, wherein an adhesive is also coated on another face of the support.

6. An analysis device comprising a rigid support comprising at least one well delimiting an internal cavity comprising at least two apertures, at least one of said apertures of the cavity being sealed by the complex as claimed in claim 1.

7. The device as claimed in claim 6, chosen from analysis plates.

8. A method comprising preparing an analysis device as claimed in claim 6.

9. A process for preparing a complex as claimed in claim 1, comprising the following steps:
a) dilution of the ligand in a suitable solution, for example a saline buffer;
b) deposition of the ligand in solution onto the surface of the adhesive.

10. The process for preparing as claimed in claim 9, comprising a step of pretreatment of the adhesive surface.

11. The process for preparing a complex as claimed in claim 9, comprising an additional step c) of drying the liquid thus deposited.

12. The process for preparing as claimed in claim 9, further comprising an additional step d) of heating, washing and/or saturation.

13. A process comprising preparing a device as claimed in claim 6, by sealing the complex with the support so as to close at least one of said apertures of the cavity, the adhesive surface comprising the ligand being oriented toward the interior of the cavity.

14. A process for detecting and/or quantifying an antiligand, comprising the steps:
a) placing the device as claimed in claim 6 in contact with a sample liable to contain an antiligand under conditions enabling interaction between the ligand and the antiligand,
b) optionally, adding a labeled detection molecule,
c) detection and/or quantification of the signals generated by the interaction between the ligand and the antiligand.

15. The process for detecting and/or quantifying an antiligand as claimed in claim 14, in which the antiligand originates from a biological sample, an environmental sample or a sample originating from the agrifood industry.

16. The process for detecting and/or quantifying an antiligand as claimed in claim 14, by colorimetry, fluorescence, chemiluminescence, radiography or detection of radioactivity.
